(19) United States
(12) Patent Application Publication
(10) Pub. No.: US 2026/0269035 A1
(43) Pub. Date: Sep. 10, 2026

(54) MEDICAL DIAGNOSIS SYSTEM BASED ON ARTIFICIAL INTELLIGENCE CHATBOT AND OPERATING METHOD THEREFOR

(71) Applicant: IDSSET Inc., Wonju-si (KR)

(72) Inventors: Young Seop Hong, Wonju-si (KR); Tae Ik Kwon, Wonju-si (KR)

(21) Appl. No.: 19/277,040

(22) Filed: Jul. 22, 2025

(30) Foreign Application Priority Data
Mar. 4, 2025 (KR) 10-2025-0027252

(51) Int. Cl. G16H 15/00; G06Q 10/02; G16H 10/20; G16H 40/67

(52) U.S. Cl. CPC G16H 15/00; G06Q 10/02; G16H 10/20; G16H 40/67

(57) ABSTRACT

An operating method for an interactive medical chatbot-based hospital reservation and diagnosis assistance system performed by a server device according to the present embodiment includes: generating pre-questionnaire information including a plurality of status confirmation comments for identifying a health status of a patient from one or more user terminals associated with the patient when the one or more user terminals associated with the patient access an interface for the interactive medical chatbot; receiving a plurality of status answer comments in response to the pre-questionnaire information from the one or more user terminals associated with the patient; generating symptom prediction information for the patient based on the plurality of status answer comments, wherein the symptom prediction information is associated with an output response of a pre-trained lightweight LLM module; transferring a hospital reservation link including hospital reservation information that indicates one or more hospitals available for reservation when a hospital reservation is determined to be necessary based on the symptom prediction information; and transferring symptom summary information associated to the patient and predicted prescription information based on the symptom prediction information to at least one hospital desired for visit among the one or more hospitals available for reservation, wherein the symptom summary information includes information summarized by the lightweight LLM module based on the pre-questionnaire information and the plurality of status answer comments between the patient and the interactive medical chatbot.

### CROSS-REFERENCE TO RELATED APPLICATIONS

This application claims the priority of the Korean Patent Applications NO 10-2025-0027252, filed on Mar. 4, 2025, in the Korean Intellectual Property Office. The entire disclosures of all these applications are hereby incorporated by reference.

### BACKGROUND OF THE INVENTION

### 1. Field of the Invention

The present specification relates to a hospital reservation and diagnosis assistance system based on an interactive medical chatbot and an operating method therefor, and more particularly, an artificial intelligence chatbot-based medical diagnosis system and an operating method therefor to skip general and repetitive questions from medical staff and corresponding answers from a patient in the conventional questionnaire process by implementing a pre-questionnaire function based on generative artificial intelligence, so that the efficiency of treatment time can be remarkably improved.

### 2. Description of the Related Art

In modern society, accessibility and quality of medical services are positioned as essential elements for maintaining a healthy life. However, since medical facilities and medical staff are mainly concentrated in large cities, the imbalance in accessibility to medical services between regions is becoming a serious problem. This medical imbalance has been increasingly worse especially due to aging and population growth. It is difficult to access adequate medical services in rural and provincial areas, which exerts a negative influence on overall national health.

In addition, it is needed to improve schemes on medical data management. Since most medical institutions still use paper-based medical records, there is a limit in management and utilization of information. In order to solve the above problems, Vietnam aims to convert records on 95% of its population to electronic health records by 2025, and this can be seen as an example that emphasizes the need for digital medical data management.

Meanwhile, attempts to improve accessibility by utilizing technology at all stages of a medical services have attracted attention. Approaches have been developed to remotely handle all stages of medical services, including checkups, treatment, and prescriptions in addition to home care. This enables patients to receive medical services at home without visiting hospitals, and it may be a useful solution especially for small rural hospitals and residents living in provincial areas. In order to implement the remote medical services, it is also expected that the free distribution of basic test equipment and disease management medical appliances to households and small rural hospitals may play an important role.

Recently, artificial intelligence-based technologies have played an important role in the medical service field. Particularly, artificial intelligence-based medical chatbots analyze symptoms and diseases of patients and provide appropriate information, thereby contributing to reducing the burden on medical staff and increasing early access to medical services of patients. These chatbots may utilize natural language processing and machine learning algorithms to interact with users, and provide simple diagnostic and counseling services. Accordingly, the chatbots remarkably contribute to increasing the efficiency of medical services.

In addition, the development of lightweight large language model (LLM) modules provides a significant turning point in technological advancement in the medical field. The lightweight LLM module has the ability to analyze large amounts of data and process the data in real time while minimizing requirements for computing resources, thereby allowing utilization in small medical institutions or remote environments. Since these technologies may be utilized for real-time analysis of medical data, patient-customized diagnosis, and treatment planning, it is expected to contribute to further improving the quality of medical services.

For the present specification, Korean Patent Application No. 10-2022-0113186 entitled by “MEDICAL DIAGNOSIS SYSTEM BASED ON ARTIFICIAL INTELLIGENCE AND OPERATION METHOD THEREOF” may be referred.

### SUMMARY OF THE INVENTION

An object of the present specification is to provide an artificial intelligence chatbot-based medical diagnosis system and an operating method therefor to replace general and repetitive questions from medical staff and corresponding answers from a patient in the conventional questionnaire process by implementing a pre-questionnaire function based on generative artificial intelligence, so that the efficiency of treatment time is remarkably improved.

In addition, an object of the present specification is to provide an artificial intelligence chatbot-based medical diagnosis system and an operating method therefor compared to the conventional scenario-based chatbots having limitations in responding to various questions from patients, to quickly generate optimized responses by mounting a lightweight LLM (that is, smaller Large Language Model, hereinafter referred to as ‘sLLM’) module, and perform real-time feedbacks and improvements on outputs (e.g., diagnosis) of the lightweight LLM module by introducing a feedback loop.

The technical problems to be achieved through the present specification are not limited to the above-mentioned technical problems, and other technical problems not mentioned will be clearly understood by a person having ordinary skill in the art to which the present specification belongs from the description below.

An operating method for an interactive medical chatbot-based hospital reservation and diagnosis assistance system performed by a server device according to the present embodiment includes: generating pre-questionnaire information including a plurality of status confirmation comments for identifying a health status of a patient from one or more user terminals associated with a patient when the one or more user terminals associated with the patient access an interface for the interactive medical chatbot; receiving a plurality of status answer comments in response to the pre-questionnaire information from the one or more user terminals associated with the patient; generating symptom prediction information for the patient based on the status answer comments, in which the symptom prediction information is associated with an output response of a pre-trained lightweight LLM module; transferring a hospital reservation link including hospital reservation information that indicates one or more hospitals available for reservation when a hospital reservation is determined to be necessary based on the symptom prediction information; and transferring symptom summary information associated to the patient and predicted prescription information based on the symptom prediction information to at least one hospital desired for visit among the hospitals available for reservation, in which the symptom summary information includes information summarized by the lightweight LLM module based on the pre-questionnaire information and the status answer comments between the patient and the interactive medical chatbot.

The interactive medical chatbot-based hospital reservation and diagnosis assistance system according to the present embodiment includes: one or more user terminals; and a server device linked to the alt least one user terminal, wherein the server device generates pre-questionnaire information including a plurality of status confirmation comments for identifying a health status of a patient from one or more user terminals associated with a patient when the one or more user terminals associated with the patient access an interface for the interactive medical chatbot; receives a plurality of status answer comments in response to the pre-questionnaire information from the one or more user terminals associated with the patient; generates symptom prediction information for the patient based on the status answer comments, in which the symptom prediction information is associated with an output response of a pre-trained lightweight LLM module; transfers a hospital reservation link containing hospital reservation information indicating one or more hospitals available for reservation when a hospital reservation is determined to be necessary based on the symptom prediction information; and transfers symptom summary information associated to the patient and predicted prescription information based on the symptom prediction information to at least one hospital desired for visit among the hospitals available for reservation, in which the symptom summary information includes information summarized by the lightweight LLM module based on the pre-questionnaire information and the status answer comments between the patient and the interactive medical chatbot.

According to the technical solution mentioned in the present specification, an artificial intelligence chatbot-based medical diagnosis system and an operating method therefor can be provided to replace general and repetitive questions from medical staff and corresponding answers from a patient in the conventional questionnaire process by implementing a pre-questionnaire function based on generative artificial intelligence, so that the efficiency of treatment time can be remarkably improved.

In addition, according to the technical solution mentioned in the present specification, an artificial intelligence chatbot-based medical diagnosis system and an operating method therefor can be provided optimized answers can be quickly generated by mounting a lightweight LLM module compared to the conventional scenario-based chatbots having limitations in responding to various questions from patients, and real-time feedbacks and improvements on outputs (e.g., diagnosis) of the lightweight LLM module can be performed by introducing a feedback loop.

Meanwhile, it will be understood that the present specification is not limited to the above-described advantageous effects, and includes all effects that can be inferred from the configuration of the invention described in the detailed description or the claims.

### BRIEF DESCRIPTION OF THE DRAWINGS

FIG. 1 is a conceptual diagram showing an interactive medical chatbot-based hospital reservation and diagnosis assistance system according to the present embodiment.

FIG. 2 is a block diagram showing an interactive medical chatbot server-implemented device according to the present embodiment.

FIG. 3 is a flowchart showing an operating method for the medical chatbot-based hospital reservation and diagnosis assistance system according to the present embodiment.

FIG. 4 is a flowchart showing an operating method for the interactive medical chatbot-based hospital reservation and diagnosis assistance system according to the present embodiment.

FIG. 5 is a flowchart for explaining a process of accurately generating predicted prescription information of the operating method for the interactive medical chatbot-based hospital reservation and diagnosis assistance system according to the present embodiment.

FIG. 6 is a screen example exemplarily showing a conversation process provided through an interactive medical chatbot interface according to the one embodiment.

FIG. 7 shows an exemplary interface result screen for providing symptom prediction information to a user terminal according to the operation of the interactive medical chatbot of the one embodiment.

FIG. 8 shows an exemplary interface screen of a reservation link screen according to the operation of the interactive medical chatbot of the one embodiment.

FIG. 9 shows an exemplary interface screen in which a conversation about symptoms between a user and the interactive medical chatbot according to the one embodiment is automatically summarized.

FIG. 10 is a block diagram for explaining a computing environment including a computing device suitable for use in an exemplary embodiment.

### DETAILED DESCRIPTION OF THE INVENTION

Advantages and features of the present invention, and methods for achieving the advantages and features will be apparent with reference to the embodiments described below in detail with the accompanying drawings. However, the present invention is not limited to the embodiments disclosed below and may be implemented in various different forms, and these embodiments are provided only to make the disclosure of the present invention complete and to fully inform a person having ordinary skill in the art of the scope of the present invention, and the present invention is defined only by the scope of the claims.

The terms used herein are for the purpose of describing embodiments and are not intended to limit the present invention. Herein, a singular form includes a plural form unless the context is particularly stated otherwise. The expressions “comprises” and/or “comprising” used herein do not exclude the presence or addition of another element other than the mentioned elements. Throughout the specification, like reference numerals refer to like elements, and “and/or” includes each of the mentioned elements or one or more combinations thereof. Although the terms “first”, “second” and the like are used to describe various elements, the elements are not limited by these terms. The above terms are used merely to distinguish one element from the other elements. Therefore, it will be understood that the first element mentioned below may be the second element within the technical idea of the present invention.

Unless otherwise defined, all terms (including technical and scientific terms) used herein may be used in a sense commonly understood by a person having ordinary skill in the art of the present invention. Further, terms defined in generally used dictionaries are not ideally or excessively interpreted unless explicitly defined otherwise.

In this specification, the ‘sLLM module’ refers to a lightweight large language model (LLM), and hereinafter, may also be referred to as a ‘lightweight LLM module’.

Hereinafter, an interactive medical chatbot-based hospital reservation and diagnosis assistance system according to the present embodiment will be described with reference to the drawings.

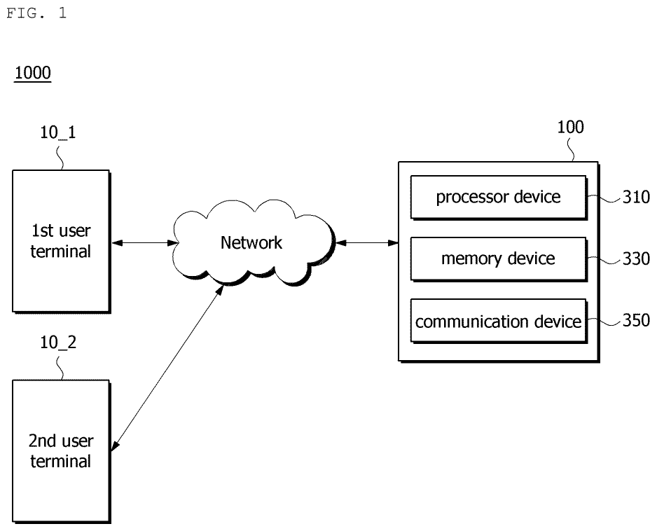

FIG. 1 is a conceptual diagram of an interactive medical chatbot-based hospital reservation and diagnosis assistance system according to the present embodiment.

Referring to FIG. 1, an interactive medical chatbot-based hospital reservation and diagnosis assistance system 1000 according to the present embodiment may include one or more user terminals 10_1 and 10_2 and an interactive medical chatbot-implemented server device 100.

The one or more user terminals 10_1 and 10_2 of FIG. 1 may access the interactive medical chatbot-implemented server device 100 and located in a remote site by using a pre-established wireless network NW.

For example, a user (e.g., a patient) may access the server device 100 via the wireless network NW by using a first user terminal 10_1. In addition, a user (e.g., medical staff) may access the server device 100 via the wireless network NW by using a second user terminal 10_2.

In this case, the one or more user terminals 10_1 and 10_2 may be provided with various medical services or medical information while conversing with an interactive medical chatbot while being connected to an interactive medical chatbot interface pre-implemented in the server device 100.

As an example, the one or more user terminals 10_1 and 10_2 may correspond to devices to which IP addresses are assigned, and perform network communication via the Internet or the like.

The one or more user terminals 10_1 and 10_2 refer to a wireless communication device that ensures portability and mobility, and may include all kinds of wireless communication devices such as navigation, personal communication system (PCS), global system for mobile communications (GSM), personal digital cellular (PDC), personal handy-phone system (PHS), personal digital assistant (PDA), international mobile telecommunication (IMT)-2000, code division multiple access (CDMA)-2000, W-code division multiple access (W-CDMA), wireless broadband internet (Wibro) terminal, smartphone, smartpad, tablet PC, and may include wired communication devices such as desktop, but it will be understood that the present invention is not limited thereto.

Meanwhile, it will be understood that the one or more user terminals 10_1 and 10_2 are not limited to the above-described types, and may include any device capable of communicating with external devices.

For example, the one or more user terminals 10_1 and 10_2 may be understood as terminals on which an application (APP) associated with the interactive medical chatbot is installed or mounted in advance.

As an example, a user may access the server device 100 through a medical application installed or mounted in advance on the one or more user terminals 10_1 and 10_2, thereby conversing with the interactive medical chatbot implemented in the server device 100, so that various medical services may be provided.

The wireless network NW of FIG. 1 may be understood as a connection structure for implementing information exchanges by multiple wireless nodes between the one or more user terminals 10_1 and 10_2 and the interactive medical chatbot-implemented server device 100.

For example, it will be understood that a pre-established wireless communication protocol for wireless network NW may include, but is not limited to, RF, 3rd generation partnership project (3GPP) network, long term evolution (LTE) network, 5th generation partnership project (5GPP) network, world interoperability for microwave access (WIMAX) network, Internet, local area network (LAN), wireless local area network (Wireless LAN), wide area network (WAN), personal area network (PAN), Bluetooth network, NFC network, satellite broadcasting network, analog broadcasting network, digital multimedia broadcasting (DMB) network, and the like.

The interactive medical chatbot-implemented server device 100 of FIG. 1 may provide various medical services or medical information to users while conversing through the interactive medical chatbot with the one or more user terminals 10_1 and 10_2 connected via the wireless network NW.

For example, The interactive medical chatbot-implemented server device 100 of FIG. 1 may be a device managed (or operated) by an administrator of an interactive medical chatbot pre-established on the server device 100.

Meanwhile, the interactive medical chatbot-implemented server device 100 may include a processor device 110, a memory device 120, and a communication device 130.

For example, the processor device 110 is implemented based on hardware and software that performs natural language processing, and may be understood as a configuration that controls not only the operation of software for the interactive medical chatbot, but also the implementation of input/output and overall functions of the server device 100.

More particularly the processor device 110 may include one or more application processors AP, one or more communication processors CP, or one or more artificial intelligence (AI) processors. The application processor, the communication processor, or the AI processor may be contained within different integrated circuit (IC) packages, respectively, or within a single IC package.

The application processor may drive an operating system or application program to control a plurality of hardware or software components connected to the application processor, and perform various data processing/operations, including multimedia data. As an example, the application processor may be implemented as a system on chip (SoC). The processor 310 may further include a graphic processing unit (GPU).

The communication processor may perform functions of managing data links and converting communication protocols in communication with the user device 100 connected via network. As an example, the communication processor may be implemented as an SoC. The communication processor may perform at least some of the multimedia control functions.

In addition, the communication processor may control data transmission and reception of the communication module 350. The communication processor may be implemented to be included as at least a part of the application processor.

For the reference, the application processor or the communication processor may be implemented to load instructions or data received from at least one of non-volatile memories or other components connected thereto into the volatile memory and process the loaded instructions or data.

In addition, the application processor or the communication processor may be implemented to store data received from or generated by at least one of the other components into a nonvolatile memory.

For example, the memory device 120 may be understood as a configuration that corresponds to a pre-established database and stores various types of data acquired from outside or within the system, as well as stores and manages basic source code for a lightweight LLM module of an interactive medical chatbot and various related datasets for advancement of the lightweight LLM module.

More particularly the memory device 120 may include a built-in memory or an external memory. The built-in memory may include at least one of volatile memory (such as dynamic RAM (DRAM), static RAM (SRAM) and synchronous dynamic RAM (SDRAM)) or non-volatile memory (such as one time programmable ROM (OTPROM), programmable ROM (PROM), erasable and programmable ROM (EPROM), electrically erasable and programmable ROM (EEPROM), mask ROM, flash ROM, NAND flash memory, and NOR flash memory). As an example, the built-in memory may take the form of a solid state drive (SSD). The external memory may include a flash drive, such as compact flash (CF), secure digital (SD), micro secure digital (Micro-SD), mini secure digital (Mini-SD), extreme digital (XD), or a memory stick.

For example, the communication device 130 may include a wireless communication module or an RF module. As an example, the wireless communication module may include Wi-Fi, BT, GPS circuitry or NFC circuitry. The wireless communication module may provide wireless communication functions using radio frequencies.

The wireless communication module may be implemented to include a network interface, a modem or the like for connecting the one or more user terminals 10_1 and 10_2 to the network (such as Internet, LAN, WAN, telecommunication network, cellular network, satellite network, POTS or 5G network).

The RF module may be understood as a configuration for transmitting and receiving data, for example, transmitting and receiving RF signals or called electronic signals. In other words, the RF module may include a transceiver, a power amp module (PAM), a frequency filter, or a low noise amplifier (LNA). In addition, the RF module may be implemented to include a part for transmitting and receiving electromagnetic waves in free space in wireless communication, such as a conductor or a wire.

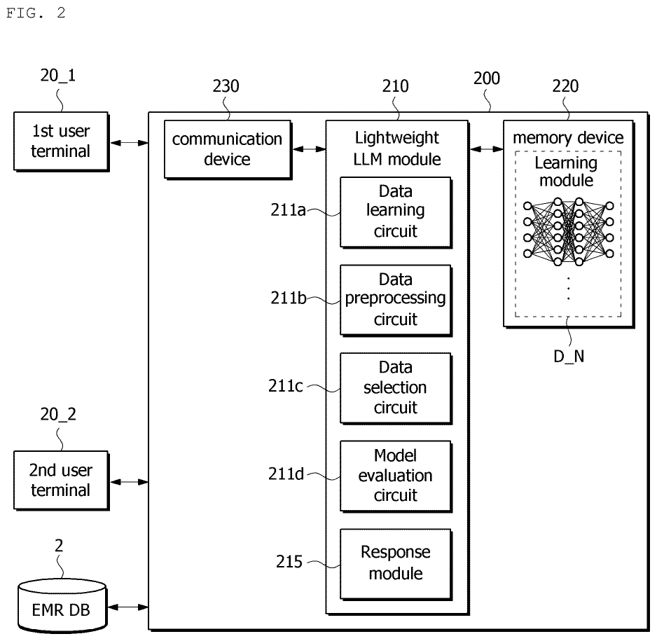

Meanwhile, for more details of the interactive medical chatbot-implemented server device 100, refer to FIG. 2 described later.

FIG. 2 is a block diagram showing the interactive medical chatbot-implemented server device according to the present embodiment.

Referring to FIGS. 1 and 2, the interactive medical chatbot-implemented server device 200 of FIG. 2 may have a configuration corresponding to the server device 100 of FIG. 1. In other words, the interactive medical chatbot-implemented server device 200 may include a processor device 210, a memory device 220, and a communication device 230.

The processor device 210 of FIG. 2 may be implemented based on an AI-dedicated processor (e.g., GPU) for artificial intelligence learning for implementing an interactive medical chatbot. The processor device 210 according to the present embodiment may include a lightweight LLM module 211 and a response module 215.

For example, the lightweight LLM module 211 may include a data learning circuit 211a, a data preprocessing circuit 211b, a data selection circuit 211c, and a model evaluation circuit 211d.

As an example, the data learning circuit 211a may learn criteria regarding which learning data to use to determine data classification/recognition and how to classify and recognize data using the learning data in order to train a neural network model for data classification/recognition. The data learning circuit 211a may be understood as a configuration that acquires learning data to be used for learning and performs learning by applying the acquired learning data to a deep learning model.

Additionally, the data learning circuit 211a may be manufactured in the form of at least one hardware chip and mounted on the interactive medical chatbot-implemented server device 200. As an example, the data learning circuit 211a may be manufactured in the form of a dedicated hardware chip for artificial intelligence or manufactured as a portion of a general-purpose processor (CPU) or a graphics-only processor (GPU) and mounted on the interactive medical chatbot-implemented server device 200.

In addition, the data learning circuit 211a may also be implemented as a software module. When it being implemented as a software module (or a program module containing instructions), the software module may be stored in a non-transitory computer readable medium that can be read by a computer. In this case, at least one software module may be provided to an operating system (OS) or provided by an application.

The data learning circuit 211a may train learn to have a determination criterion for allowing a neural networks model to classify/recognize predetermined data by using the acquired learning data.

In this case, the learning method by the data learning circuit 211a may be classified into supervised learning, unsupervised learning, or reinforcement learning.

The supervised learning refers to a method of training an artificial neural network when labels for learning data are given, and the label may signify a correct answer (or result value) that the artificial neural network is required to infer when the learning data is input to the artificial neural network. The unsupervised learning may refer to a method of training an artificial neural network without being given labels for the learning data. The reinforcement learning may refer to a method that trains an agent defined in a specific environment to select actions or action sequences that maximize cumulative rewards in each state. In addition, the data learning circuit 211a may train a neural network model using a learning algorithm including error backpropagation or gradient descent.

The neural network model (that is, neural network) may be associated with at least one of a bidirectional encoder representations from transformers (BERT) model, a DistilBERT model, a TinyBERT model, a lite BERT (ALBERT) model, a MobileBERT model, and a GPT-NeoX/TinyGPT model.

According to the present specification, when the neural network model is trained beyond a pre-determined threshold level, the trained neural network model may be referred to as a learning model D_N within the present specification. The learning model D_N may be stored in the memory device 220 and may be called from the processor device 210 toward the processor device 210 to infer results for new input data other than previously learned data.

More particularly, the processor device 210 may further include a data preprocessing circuit 211b, a data selection circuit 211c, and a model evaluation circuit 211d to improve analysis results using the learning model D_N or to save resources or time required for generating the learning model D_N.

As an example, the data preprocessing circuit 211b may be implemented to preprocess the acquired data so that the acquired data may be used for learning/inference for situational determination. As an example, the data preprocessing circuit 211b may be implemented to extract feature information by performing preprocessing on input data acquired through the one or more user terminals 20_1 and 20_2. The feature information may be extracted as a format such as feature vector, feature point, or feature map.

The data selection circuit 211c may be implemented to select data for the learning model D_N from among the learning data preprocessed in the above-described data learning circuit 211a or data preprocessing circuit 211b.

The model evaluation circuit 211d may be implemented to input evaluation data into the neural network model, and allow the data learning circuit 211a to perform learning again when the analysis results output from the evaluation data do not satisfy a predetermined criterion.

In this case, the evaluation data may be preset data for evaluating the learning model D_N. As an example, the model evaluation circuit 211d may be implemented to evaluate that the predetermined criterion is not satisfied when the number or ratio of evaluation data for which the analysis results of the trained neural network model for the evaluation data are inaccurate exceeds a preset threshold.

For example, the response module 215 may recognize data related to questions and answers with the one or more user terminals 20_1 and 20_2 based on the learning model D_N for the interactive medical chatbot, and be linked to a pre-established electronic medical record (EMR) database 2 for a hospital or medical platform desired for visit. The EMR database 2 may be linked to the server device 200 based on a predetermined communication protocol.

As an example, when the user's consent is obtained in the subscription process of an application (APP) associated with the interactive medical chatbot pre-installed or mounted on the one or more user terminals 10_1 and 10_2, medical history information of each patient may be stored and managed on the EMR database 2.

As an example, the response module 215 may be implemented to conduct questions and answers between the one or more user terminals 20_1 and 20_2 and the interactive medical chatbot under the control of the processor device 210.

As an example, the response module 215 may be implemented to generate pre-questionnaire information generated to determine a patient's health status, and provide an output response (that is, user's symptom prediction information) of the learning model D_N based on multiple status answer comments and additional answer information received from the user.

For example, the memory device 220 may store various programs and data required for operations of the one or more user terminals 20_1 and 20_2 and/or the interactive medical chatbot-implemented server device 200.

In addition, the memory device 220 may be accessed by the lightweight LLM module 211, and operations such as reading/recording/modifying/deleting/updating data may be performed by the lightweight LLM module 211 under the control of the processor device 210.

Meanwhile, the memory device 220 may store an artificial intelligence model (that is, a neural network model) generated through a learning algorithm for data classification/recognition according to one embodiment of the present specification. In addition, the memory device 220 may store not only the learning model D_N, but also input data, learning data, learning history and the like.

For example, the communication device 230 may be understood as a configuration for transmitting processing results by the processor device 210 to the one or more user terminals 20_1 and 20_2 as well as receiving user data from the one or more user terminals 20_1 and 20_2.

As an example, the one or more user terminals may include a first user terminal 20_1 associated with a patient and a second user terminal 20_2 associated with medical staff of at least one hospital desired for visit.

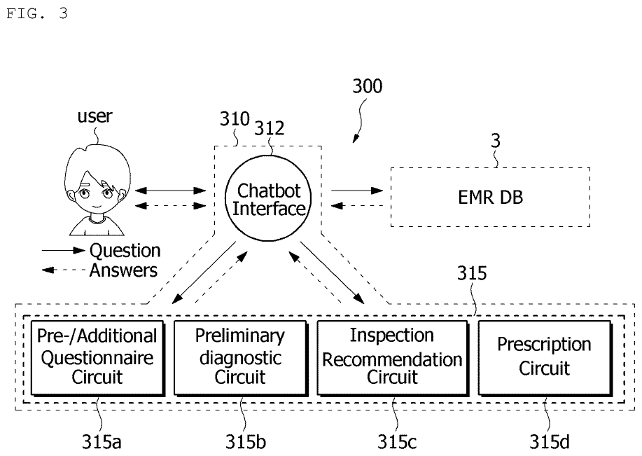

FIG. 3 is a view for explaining the structure of the response module for the interactive medical chatbot according to the present embodiment.

Referring to FIGS. 1 to 3, the processor device 310 of FIG. 3 may correspond to the above-described processor device (e.g., 210 of FIG. 2), the one or more users of FIG. 3 may be associated with the above-described one or more user terminals (e.g., 20_1 of FIG. 2), and the EMR database 3 of FIG. 3 may correspond to the above-described EMR database (e.g., 2 of FIG. 2).

The interactive medical chatbot according to the present embodiment may be implemented based on the Google Dialogflow chatbot builder to collect patient health information into an information collection module through a single multi-conversation. As an example, Google Dialogflow may be implemented to support 20 languages, including Korean, among chatbot builders, based on the Google's NLU engine.

In addition, the response module 315 of FIG. 3 may correspond to the above-described response module (e.g., 215 of FIG. 2).

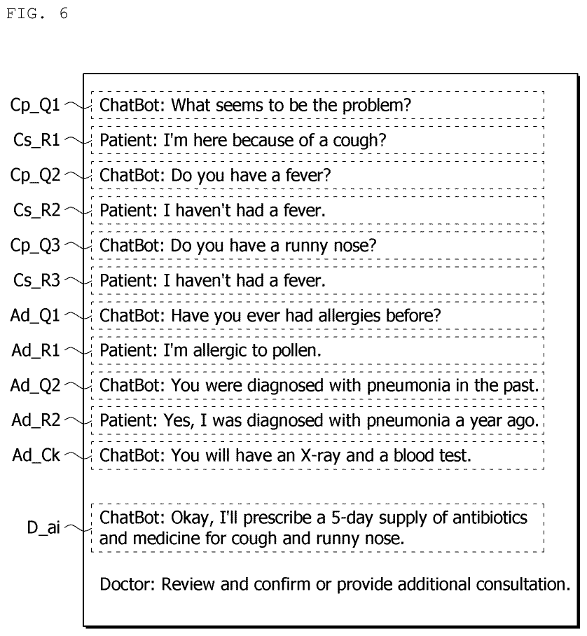

The processor device 310 according to the present embodiment may be implemented to check the questionnaire by providing one or more status confirmation comments (e.g., Cp_Q1 to Cp_Q3 of FIG. 6) to the user through the one or more user terminals (e.g., 20_1 and 20_2 of FIG. 2).

In addition, the processor device 310 may be implemented to collect patient information and/or user inquiries associated with the one or more user terminals (e.g., 20_1 of FIG. 2) based on the questionnaire checked through the multiple status answer comments (e.g., Cs_R1 to Cs_R3 of FIG. 6).

In addition, the processor device 310 may classify the user queries collected through the one or more user terminals (e.g., 20_1 and 20_2 of FIG. 2) based on the scope of pre-stored (or pre-set) general medical information, as well as may learn medical information capable of answering the users' queries based on the classified users' queries, and derive answers to the user queries based on learned medical information.

As an example, when a patient's question or inquiry is input through the chatbot interface 312, the processor device 310 may be implemented to extract the patient's intention or the patient's intention for the inquiry by analyzing and learning the patient's question or inquiry.

In addition, the processor device 310 may be implemented to not only classify the extracted patient's intention but also output an appropriate answer to the patient's inquiry.

In other words, the chatbot interface 312 may not only check and/or manage the overall conversation between the user and the interactive medical chatbot under the control of the processor device 310, but also search for and activate at least one circuit (315a to 315d) included in the response module 315 in response to the user's query.

More particularly, the chatbot interface 312 may be implemented to ask the patient, “Which part are you feeling uncomfortable?” when a chat session is started through a screen as shown in FIG. 6 with the one or more user terminals (e.g., 20_1 of FIG. 2) associated with the patient under the control of the processor 310.

Meanwhile, the response module 315 of FIG. 3 may include a pre-/additional questionnaire circuit 315a, a preliminary diagnostic circuit 315b, an inspection recommendation circuit 315c, and a preliminary prescription circuit 315d.

For example, the pre-/additional questionnaire circuit 315a may generate pre-questionnaire information to determine a health status of a user (e.g., a patient) associated with the one or more user terminals (e.g., 20_1 of FIG. 2).

The pre-questionnaire information may include one or more pre-prepared status confirmation statements (e.g., Cp_Q1 to Cp_Q3 of FIG. 6) as shown in FIG. 6 described later.

As an example, a plurality of status answer comments (e.g., Cs_R1 to Cs_R3 in FIG. 6) may be received in response to a plurality of status confirmation comments (e.g., Cp_Q1 to Cp_Q3 in FIG. 6) provided sequentially (or according to a user's response) to one or more user terminals (e.g., 20_1 in FIG. 2).

In addition, the pre-/additional questionnaire circuit 315a may determine whether the provision of the pre-prepared status confirmation comments (e.g., Cp_Q1 to Cp_Q3 of FIG. 6) is completed (that is, whether the provision of additional status confirmation comments is unnecessary).

Meanwhile, when it is determined that the provision of one or more additional confirmation comments (e.g., Ad_Q1 and Ad_Q2 in FIG. 6) is unnecessary, the pre-/additional questionnaire circuit 315a may transfer the status answer comments (e.g., Cs_R1 to Cs_R3 of FIG. 6) to the preliminary diagnostic circuit 315b.

When it is determined that it is necessary to provide one or more additional confirmation comments (e.g., Ad_Q1, Ad_Q2 in FIG. 6) to the one or more user terminals (e.g., 20_1 in FIG. 2), the pre-/additional questionnaire circuit 315a may be implemented to generate (and provide) additional questionnaire information based on the status answer comments (e.g., Cs_R1 to Cs_R3 of FIG. 6) received from the one or more user terminals (e.g., 20_1 of FIG. 2) and medical history information of the patient associated with the one or more user terminals (e.g., 20_1 of FIG. 2).

The medical history information of the user (e.g., the patient) may be implemented to be transferred from a predetermined EMR database 3 established in advance for a hospital or medical platform desired for visit to the pre-/additional questionnaire circuit 315a.

The additional questionnaire information may include one or more additional confirmation comments (e.g., Ad_Q1 and Ad_Q2 in FIG. 6) additionally generated as shown in FIG. 6 described later.

For example, the preliminary diagnostic circuit 315b may generate symptom prediction information based on medical history information of the user (e.g., the patient) associated with the one or more user terminals (e.g., 20_1 of FIG. 2), a plurality of status answer comments (e.g., Cs_R1 to Cs_R3 of FIG. 6) received from one or more user terminals (e.g., 20_1 of FIG. 2), and/or additional response information according to additional questionnaire information.

As an example, the additional answer information may include one or more additional response comments (e.g., Ad_R1, Ad_R2 in FIG. 6) that are responses to additional confirmation comments (e.g., Ad_Q1, Ad_Q2 in FIG. 6), as shown in FIG. 6 described later.

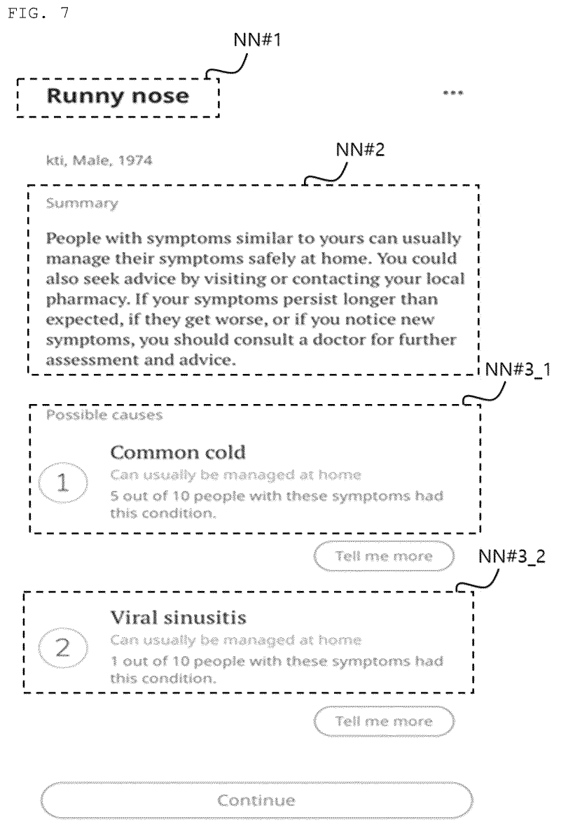

The symptom prediction information may include at least one of disease prediction information NN #1, symptom summary information NN #2, and causal factor information NN #3_1 and NN #3_2 as shown in FIG. 7 described later.

For example, the inspection recommendation circuit 315c may generate inspection recommendation information that requests performance of at least one inspection (e.g., blood test, X-ray imaging) for a user (e.g., a patient) based on a plurality of status answer comments (e.g., Cs_R1 to Cs_R3 of FIG. 6) and one or more additional answer comments (e.g., Ad_R1, Ad_R2 of FIG. 6).

As an example, the inspection recommendation information may include one or more inspection recommendation comments (e.g., Ad_Ck in FIG. 6).

In addition, when at least one hospital is reserved by the one or more user terminals (e.g., 20_1 in FIG. 2), the inspection recommendation circuit 315c may be implemented to receive inspection result information according to the patient's inspection recommendation information from the at least one reserved hospital.

For example, the preliminary prescription circuit 315d may generate predicted prescription information based on the inspection result information and the symptom prediction information. In addition, in this case, the predicted prescription information may be provided to a wireless terminal (not shown) of medical staff of the at least one reserved hospital desired for visit.

As an example, the predicted prescription information may include one or more predicted prescription comments D_ai.

Meanwhile, the preliminary prescription circuit 315d may be implemented to obtain confirmation on the one or more predicted prescription comments D_ai or information on additional treatment from the wireless terminal (e.g., 20_2 of FIG. 2) of the medical staff of the at least one reserved hospital desired for visit through the predicted prescription information.

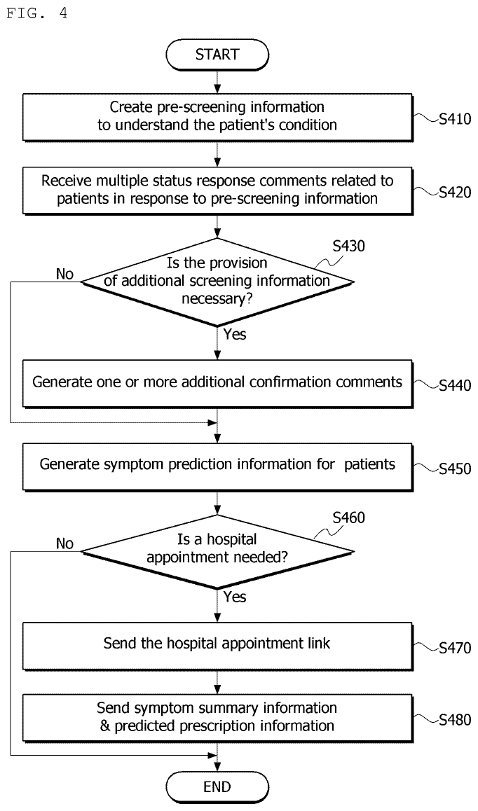

FIG. 4 is a flowchart showing an operating method for the interactive medical chatbot-based hospital reservation and diagnosis assistance system according to the present embodiment.

Referring to FIGS. 1 to 4, in step S410, when the one or more user terminals associated with the patient (e.g., 20_1 in FIG. 2) access the interface for the interactive medical chatbot (e.g., 312 in FIG. 3), the server device (e.g., 100 of FIG. 1) of the interactive medical chatbot-based hospital reservation and diagnosis assistance system (e.g., 1000 of FIG. 1) according to the present embodiment may generate pre-questionnaire information to understand a health status of a user (e.g., a patient) associated with at least one user terminal (e.g., 20_1 in FIG. 2).

For example, the pre-questionnaire information may include a plurality of status confirmation comments (e.g., Cp_Q1 to Cp_Q3 in FIG. 6).

As an example, the pre-questionnaire information including the status confirmation comments (e.g., Cp_Q1 to Cp_Q3 in FIG. 6) may be provided to the one or more user terminals (e.g., 20_1 in FIG. 2) associated with the patient.

In step S420, the server device (e.g., 100 of FIG. 1) of the interactive medical chatbot-based hospital reservation and diagnosis assistance system (e.g., 1000 of FIG. 1) according to the present embodiment may receive the status answer comments (e.g., Cs_R1 to Cs_R3 in FIG. 6) associated with the user (e.g., the patient) from the one or more user terminals (e.g., 20_1 in FIG. 2) associated with the user (e.g., the patient) in response to the pre-questionnaire information.

In step S430, the server device (e.g., 100 of FIG. 1) of the interactive medical chatbot-based hospital reservation and diagnosis assistance system (e.g., 1000 of FIG. 1) according to the present embodiment may determine whether additional questionnaire information is necessary based on the status answer comments (e.g., Cs_R1 to Cs_R3 in FIG. 6) associated with the user (e.g., the patient) and the medical history information of the user (e.g., the patient).

As an example, the patient's medical history information may be called from the predetermined EMR database (e.g., 2 in FIG. 2) to the server device (e.g., 200 in FIG. 2).

When it is determined that additional information is unnecessary, the procedure proceeds to step S450, and when it is determined that the provision of additional information is necessary, the procedure proceeds to step S440.

In step S440, the server device (e.g., 100 of FIG. 1) of the interactive medical chatbot-based hospital reservation and diagnosis assistance system (e.g., 1000 of FIG. 1) according to the present embodiment may generate one or more additional confirmation comments (e.g., Ad_Q1, Ad_Q2 in FIG. 6) based on the status answer comments (e.g., Cs_R1 to Cs_R3 in FIG. 6) and the medical history information of the user (e.g., the patient).

For example, the one or more additional confirmation comments (e.g., Ad_Q1, Ad_Q2 in FIG. 6) may be provided to the one or more user terminals (e.g., 20_1 in FIG. 2) associated with the patient.

In step S450, the server device (e.g., 100 of FIG. 1) of the interactive medical chatbot-based hospital reservation and diagnosis assistance system (e.g., 1000 of FIG. 1) according to the present embodiment may generate symptom prediction information for the user (e.g., the patient) based on the status answer comments (e.g., Cs_R1 to Cs_R3 in FIG. 6).

For example, the symptom prediction information may be associated with the output response of the response module (e.g., 215 in FIG. 2) that uses the lightweight LLM module (e.g., 211 in FIG. 2) based on a pre-trained learning model (e.g., D_N in FIG. 2).

In addition, when step S440 has proceeded, the symptom prediction information may be generated based on the status answer comments (e.g., Cs_R1 to Cs_R3 in FIG. 6) and the one or more additional answer comments (e.g., Ad_R1 and Ad_R2 in FIG. 6).

As an example, the symptom prediction information generated by the response module (e.g., 215 in FIG. 2) using the lightweight LLM module (e.g., 211 in FIG. 2) based on the pre-trained learning model (e.g., D_N in FIG. 2) may include at least one of the disease prediction information (e.g., NN #1 in FIG. 7), the symptom summary information (e.g., NN #2 in FIG. 7) and the causal factor information (e.g., NN #3_1 and NN #3_2 in FIG. 7).

In step S460, the server device (e.g., 100 of FIG. 1) of the interactive medical chatbot-based hospital reservation and diagnosis assistance system (e.g., 1000 of FIG. 1) according to the present embodiment may determine whether a hospital reservation is necessary for the user (e.g., the patient) based on the symptom prediction information.

When the user's (e.g., patient's) symptoms are determined to be insignificant based on the symptom prediction information, the procedure may be closed together with a message, which recommends a rest, without providing a link for making a separate hospital reservation.

Meanwhile, when it is determined that the hospital reservation is necessary to treat the symptoms of the user (e.g., the patient) based on the symptom prediction information, the procedure proceeds to step S470.

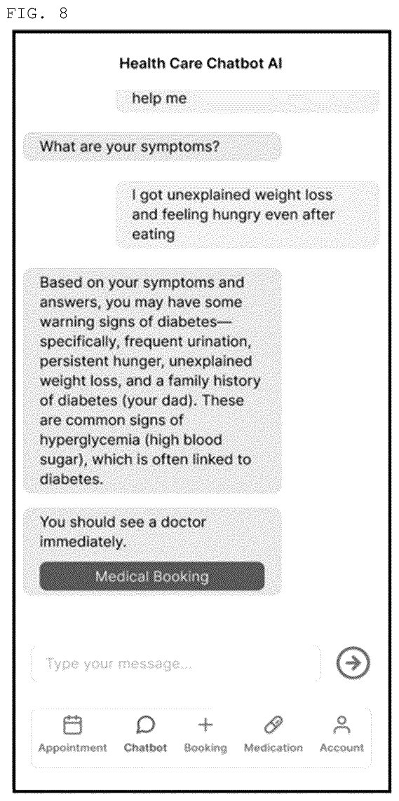

In step S470, the server device (e.g., 100 of FIG. 1) of the interactive medical chatbot-based hospital reservation and diagnosis assistance system (e.g., 1000 of FIG. 1) according to the present embodiment may provide a link for making a hospital reservation during a conversation with the interactive medical chatbot as shown in FIG. 8 described later.

For example, hospital reservation information indicating one or more hospitals available for reservations in real time and adjacent to the one or more user terminals (e.g., 10_1 of FIG. 1) associated with the user (e.g., the patient) may be provided to the one or more user terminals (e.g., 10_1 of FIG. 1) based on the symptom prediction information.

For a clear and concise understanding of the present specification, the user (e.g., the patient) may fill out reservation information through the link for hospital reservation to transfer the reservation information to the hospital desired for visit among the one or more hospitals available for reservation in real time, and then assume that the reservation has been approved by the hospital desired for visit.

In step S480, the server device (e.g., 100 of FIG. 1) of the interactive medical chatbot-based hospital reservation and diagnosis assistance system (e.g., 1000 of FIG. 1) according to the present embodiment may transfer the symptom summary information and the predicted prescription information to the one or more user terminals (e.g., 10_1 and 10_2 in FIG. 1) associated with the hospital desired for visit.

For example, the symptom summary information may be understood as information summarized by the lightweight LLM module (e.g., 211 of FIG. 2) regarding chat contents (that is, Cp_Q1 to Cp_Q3 Ad_Q1, Ad_Q2, Ad_Ck, Cs_R1, Cs_R2, Cs_R3, Ad_R1, and Ad_R2 of FIG. 6) between the user (e.g., the patient) and the interactive medical chatbot, as shown in FIG. 6 described later.

For example, the predicted prescription information may be implemented to include one or more predicted prescription comments D_ai inferred by the lightweight LLM module (e.g., 211 of FIG. 2) based on the chat contents (that is, Cp_Q1 to Cp_Q3 Ad_Q1, Ad_Q2, Ad_Ck, Cs_R1, Cs_R2, Cs_R3, Ad_R1, and Ad_R2 of FIG. 6) between the user (e.g., the patient) and the interactive medical chatbot, as shown in FIG. 6 described later.

According to the present embodiment, because the symptom summary information and the predicted prescription information for the user (e.g., the patient) may be transferred in advance to at least one user terminal (e.g., 10_2 in FIG. 1) of the hospital desired for visit, the questionnaire process repeatedly performed by the medical staff of the at least one hospital desired for visit may be simplified (or omitted), so that the patient care and management may be more efficiently performed.

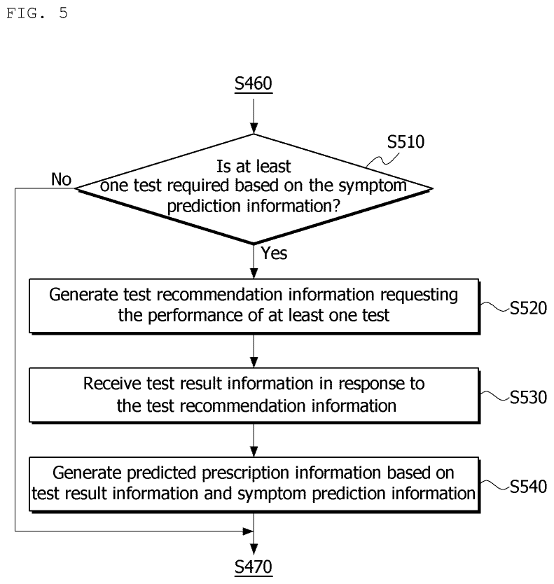

FIG. 5 is a flowchart for explaining a process of accurately generating predicted prescription information of the operating method for the interactive medical chatbot-based hospital reservation and diagnosis assistance system according to the present embodiment.

Referring to FIGS. 1 to 5, steps S510 to S540 of FIG. 5, when it is determined that the hospital reservation is necessary in step S460 of the aforementioned FIG. 4, may be understood as steps additionally performed by the server device (e.g., 100 of FIG. 1) of the interactive medical chatbot-based hospital reservation and diagnosis assistance system (e.g., 1000 of FIG. 1) according to the present embodiment.

In step S510, the server device (e.g., 100 of FIG. 1) of the interactive medical chatbot-based hospital reservation and diagnosis assistance system (e.g., 1000 of FIG. 1) according to the present embodiment may determine whether at least one inspection is necessary based on the symptom prediction information.

When the symptom is determined as being unnecessary for the at least one inspection based on the symptom prediction information, the procedure is closed (that is, immediately proceeds to step S470 of FIG. 4). Meanwhile, when the symptom is determined as being necessary for the at least one inspection based on the symptom prediction information, the procedure proceeds to step S520.

In step S520, the server device (e.g., 100 of FIG. 1) of the interactive medical chatbot-based hospital reservation and diagnosis assistance system (e.g., 1000 of FIG. 1) according to the present embodiment may generate inspection recommendation information that requests execution of the at least one inspection for the user (e.g., the patient).

The at least one inspection may include a usual inspection scheme, such as a blood inspection or X-ray inspection of the patient.

In step S530, the server device (e.g., 100 of FIG. 1) of the interactive medical chatbot-based hospital reservation and diagnosis assistance system (e.g., 1000 of FIG. 1) according to the present embodiment may receive patient's inspection result information in response to the inspection recommendation information.

For example, the inspection result information may be received by the one or more user terminals (e.g., 20_1 of FIG. 2) of the user (i.e., the patient) through the at least one user terminal (e.g., 20_2 of FIG. 2) of the at least one hospital desired for visit.

As an example, the inspection result information may include inspection results according to at least one inspection (e.g., blood inspection, X-ray inspection) performed on the user (i.e., the patient) by medical staff of at least one hospital desired for visit through the one or more user terminals (e.g., 20_1 of FIG. 2).

In step S540, the server device (e.g., 100 of FIG. 1) of the interactive medical chatbot-based hospital reservation and diagnosis assistance system (e.g., 1000 of FIG. 1) according to the present embodiment may generate predicted prescription information based on the inspection result information and the symptom prediction information.

As an example, the predicted prescription information including one or more predicted prescription comments (e.g., D_ai in FIG. 6) may be provided to the one or more user terminals associated with the patient (e.g., 20_1 in FIG. 2) and at least one user terminal associated with medical staff of at least one hospital (e.g., 20_2 in FIG. 2).

Meanwhile, in the present specification, feedback information based on actual determination of medical staff regarding the predicted prescription information may be used for re-learning of the lightweight LLM module (e.g., 211 in FIG. 2) of the server device (e.g., 200 in FIG. 2).

In addition, the patient's medical history information may be updated based on the feedback information based on the actual determination of the medical staff in a predetermined EMR database (e.g., 2 in FIG. 2) provided for each patient.

Further, it will be understood that the patient's medical history information stored and updated in the predetermined EMR database (e.g., 2 in FIG. 2) may be transferred from the EMR database (e.g., 2 in FIG. 2) upon a call from the server device (e.g., 200 in FIG. 2).

According to the present embodiment, since general and repetitive questions from medical staff and corresponding answers from a patient in the conventional questionnaire process may be replaced by implementing a pre-questionnaire function based on generative artificial intelligence, the efficiency of treatment time can be remarkably improved.

FIG. 6 is a screen example exemplarily showing a conversation process provided through the interactive medical chatbot interface according to the one embodiment.

Referring to FIGS. 1 to 6, each of multiple status confirmation comments (e.g., Cp_Q1 to Cp_Q3 of FIG. 6), one or more additional confirmation comments (e.g., Ad_Q1 and Ad_Q2 of FIG. 6), one or more inspection recommendation comments (e.g., Ad_Ck of FIG. 6), and one or more predicted prescription comments (e.g., D_ai of FIG. 6) shown in the screen example of FIG. 5 may be the output response generated by the lightweight LLM module (e.g., 211 of FIG. 2).

Meanwhile, the pre-questionnaire function of the interactive medical chatbot-based hospital reservation and diagnosis assistance system according to the present specification may also be implemented based on voice information or a photographed image on the affected area that are obtained from the user terminal (e.g., 10_1) of the user (that is, the patient).

FIG. 7 shows an exemplary interface result screen for providing symptom prediction information to a user terminal according to the operation of the interactive medical chatbot of the one embodiment.

Referring to FIGS. 1 to 7, the symptom prediction information based on the operation of the medical chatbot may be implemented to include at least one of the disease prediction information NN #1, the symptom summary information NN #2, and the causal factor information NN #3_1 and NN #3_2.

FIG. 8 shows an exemplary interface screen of a reservation link screen according to the operation of the interactive medical chatbot of the one embodiment.

Referring to FIGS. 1 to 8, a link for hospital reservation may be provided during the conversation with the interactive medical chatbot.

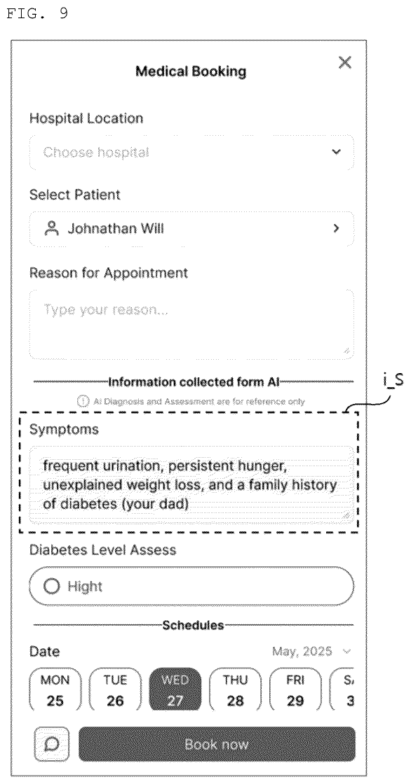

FIG. 9 shows an exemplary interface screen in which a conversation about symptoms between a user and the interactive medical chatbot according to the one embodiment is automatically summarized.

Referring to FIG. 9, the screen of FIG. 9 may be understood as a screen provided when the above-described link for hospital reservation of FIG. 8 is clicked. AI symptom summary information i_S may be provided in the screen of FIG. 9 in which a conversation between the interactive medical chatbot and the user about symptoms is automatically summarized by the lightweight LLM module (e.g., 211 of FIG. 2).

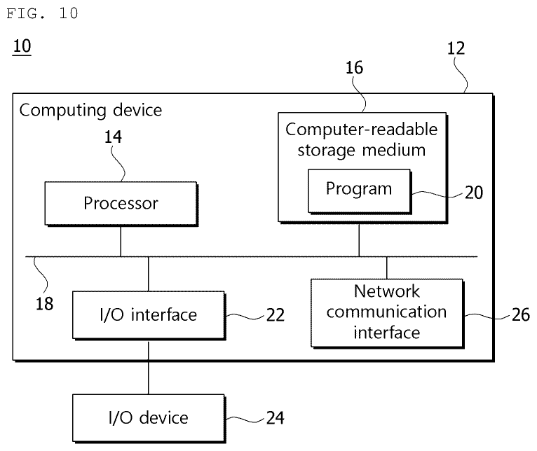

FIG. 10 is a block diagram for explaining a computing environment including a computing device suitable for use in an exemplary embodiment.

Referring to FIGS. 1 to 10, in the shown embodiments, each component may have different functions and capabilities other than those described below, and additional components other than those described below may be included.

The shown computing environment 10 includes a computing device 12. In one embodiment, the computing device 12 may correspond to the server device (e.g., 100 of FIG. 1) for the medical chatbot-based hospital reservation and diagnosis assistance system (e.g., 1000 of FIG. 1) mentioned in the present specification.

The computing device 12 includes at least one processor 14, a computer-readable storage medium 16, and a communication bus 18. The processor 14 may allow the computing device 12 to operate according to the exemplary embodiments mentioned above.

For example, the processor 14 may execute one or more programs stored in the computer-readable storage medium 16. The one or more programs may include one or more computer-executable instructions, and the one or more computer-executable instructions may be configured to allow the computing device 12 to perform operations according to the exemplary embodiments when being executed by the processor 14.

The computer-readable storage medium 16 is configured to store computer-executable instructions or program code, program data, and/or other suitable forms of information. The program 20 stored in the computer-readable storage medium 16 includes a set of instructions executable by the processor 14.

In one embodiment, the computer-readable storage medium 16 may include a memory (such as a volatile memory such as random access memory, a nonvolatile memory, or a suitable combination thereof), one or more magnetic disk storage devices, optical disk storage devices, flash memory devices, any other form of storage medium accessed by the computing device 12 to store desired information, or a suitable combination thereof.

The communication bus 18 interconnects various other components of the computing device 12, including the processor 14 and the computer-readable storage media 16.

In addition, the computing device 12 may include one or more input/output interfaces 22 configured to provide interfaces for one or more input/output devices 24 and one or more network communication interfaces 26. The input/output interfaces 22 and the network communication interfaces (26) are connected to the communication bus 18.

The input/output devices 24 may be connected to other components of the computing device 12 via the input/output interfaces 22. Exemplary input/output devices 24 may include input devices such as pointing devices (such as a mouse or trackpad), keyboards, touch input devices (such as a touchpad or touchscreen), voice or sound input devices, various types of sensor devices, and/or photographing devices, and/or output devices such as display devices, printers, speakers, and/or network cards.

The exemplary input/output device 24 may be included within the computing device 12 as a component constituting the computing device 12, and may be connected with the server device (e.g., 100 of FIG. 1) for the medical chatbot-based hospital reservation and diagnosis assistance system (e.g., 1000 of FIG. 1) according to the present embodiment, based on a pre-established communication protocol as a separate device distinct from the computing device 1.

Although the embodiments of the present invention have been described with reference to the accompanying drawings, it will be apparent that a person having ordinary skill in the art may carry out various deformations and modifications within the scope without departing from the idea of the present invention. Therefore, the above-described embodiments will be understood in all respects as illustrative and not restrictive.

Although the detailed description of the present specification has described specific embodiments, various modifications are possible without departing from the scope of present specification. Therefore, the scope of the present specification will not be limited to the above-described embodiments, and will be determined not only by the claims described below but also by equivalents of the claims of the present invention.

What is claimed is:

1. A method for operating an interactive medical chatbot-based hospital reservation and diagnosis assistance system performed by a server device, the method comprising:
generating pre-questionnaire information including a plurality of status confirmation comments for identifying a health status of a patient from one or more user terminals associated with the patient when the one or more user terminals associated with the patient access an interface for the interactive medical chatbot;
receiving a plurality of status answer comments in response to the pre-questionnaire information from the one or more user terminals associated with the patient;
generating symptom prediction information for the patient based on the plurality of status answer comments,
wherein the symptom prediction information is associated with an output response of a pre-trained lightweight LLM module;
transferring a hospital reservation link including hospital reservation information that indicates one or more hospitals available for reservation when a hospital reservation is determined to be necessary based on the symptom prediction information; and
transferring symptom summary information associated to the patient and predicted prescription information based on the symptom prediction information to at least one hospital desired for visit among the one or more hospitals available for reservation,
wherein the symptom summary information includes information summarized by the lightweight LLM module based on the pre-questionnaire information and the plurality of status answer comments between the patient and the interactive medical chatbot.

2. The method of claim 1, further comprising:
determining whether additional questionnaire information is necessary based on the plurality of status answer comments and the medical history information of the patient; and
providing one or more additional confirmation comments generated based on the plurality of status answer comments and the medical history information of the patient to the one or more user terminals associated with the patient when the additional questionnaire information is determined to be provided.

3. The method of claim 2, wherein the symptom prediction information is generated based on at least one of the plurality of status answer comments and the one or more additional answer comments.

4. The method of claim 2, further comprising:
generating inspection recommendation information for requesting execution of at least one inspection for the patient based on the symptom prediction information; and
receiving inspection result information according to the inspection recommendation information from at least one user terminal associated with medical staff of the at least one hospital desired for visit,
wherein the predicted prescription information is generated based on the inspection result information and the symptom prediction information.

5. The method of claim 4, wherein the predicted prescription information is provided to the at least one user terminal associated with the patient and the at least one user terminal associated with the medical staff of the at least one hospital desired for visit.

6. The method of claim 5, wherein feedback information received from the medical staff regarding the predicted prescription information is used to re-train the lightweight LLM module.

7. The method of claim 1, wherein the symptom prediction information includes at least one of disease prediction information, symptom summary information, and causal factor information predicted by the lightweight LLM module.

8. The method of claim 1, further comprising:
determining whether the patient is an emergency patient based on the plurality of status answer comments,
wherein the symptom prediction information is generated when the patient is determined to be a general patient other than the emergency patient.

9. An interactive medical chatbot-based hospital reservation and diagnosis assistance system comprising:
one or more user terminals; and
a server device linked to the one or more user terminals, wherein
the server device:
generates pre-questionnaire information including a plurality of status confirmation comments for identifying a health status of a patient from one or more user terminals associated with the patient when the one or more user terminals associated with the patient access an interface for the interactive medical chatbot;
receives a plurality of status answer comments in response to the pre-questionnaire information from the one or more user terminals associated with the patient;
generates symptom prediction information for the patient based on the plurality of status answer comments,
wherein the symptom prediction information is associated with an output response of a pre-trained lightweight LLM module;
transfers a hospital reservation link containing hospital reservation information indicating one or more hospitals available for reservation when a hospital reservation is determined to be necessary based on the symptom prediction information; and
transfers symptom summary information associated to the patient and predicted prescription information based on the symptom prediction information to at least one hospital desired for visit among the one or more hospitals available for reservation,
wherein the symptom summary information includes information summarized by the lightweight LLM module based on the pre-questionnaire information and the plurality of status answer comments between the patient and the interactive medical chatbot.
